(19) United States
(12) Patent Application Publication
(10) Pub. No.: US 2021/0059915 A1
(43) Pub. Date: Mar. 4, 2021

(54) COSMETIC COMPOSITION HAVING A LAMELLAR NETWORK

(71) Applicant: L'OREAL, Paris (FR)

(72) Inventor: Jun LIANG, Staten Island, NY (US)

(21) Appl. No.: 17/005,424

(22) Filed: Aug. 28, 2020

Related U.S. Application Data
(63) Continuation of application No. 62/894,183, filed on Aug. 30, 2019.

(51) Int. Cl. A61K 8/34; A61K 8/41; A61K 8/88; A61Q 5/02; A61Q 5/12; A61Q 5/00

(52) U.S. Cl. CPC A61K 8/345; A61K 8/416; A61K 8/342; A61Q 5/00; A61Q 5/02; A61Q 5/12; A61K 8/88

(57) ABSTRACT

A substantially anhydrous cosmetic composition having a lamellar network including: (a) about 20 to about 84.5 wt. % of one or more glycols chosen from ethylene glycol, propylene glycol, butylene glycol, hexylene glycol, pentylene glycol, diethylene glycol, dipropylene glycol, 1,3 propanediol, polyethylene glycols, and a mixture thereof; (b) about 15 wt. % to about 50 wt. % of one or more trihydric alcohols having from 3 to 5 carbon atoms; wherein the total amount of the one or more glycols of (a) is greater than the total amount of the one or more trihydric alcohols of (b); (c) about 0.1 to about 5 wt. % of one or more cationic surfactants; (d) about 0.1 to about 5 wt. % one or more alkoxylated fatty alcohols; and (e) about 0.1 to about 10 wt. % of one or more fatty alcohols, wherein all percentages by weight are based on the total weight of the cosmetic composition.

### CROSS-REFERENCE TO RELATED APPLICATIONS

This application claims the benefit under 35 U.S.C. § 119(e) of the filing date of U.S. Provisional Patent Application No. 62/894,183, filed on Aug. 30, 2019, entitled “COSMETIC COMPOSITION HAVING A LAMELLAR NETWORK,” the entirety of which is herein incorporated by reference.

### FIELD OF THE DISCLOSURE

The instant disclosure is directed to cosmetic compositions having a lamellar network, and particularly to substantially anhydrous cosmetic compositions having a lamellar network. Aspects of the instant disclosure also relate to methods for making and using such cosmetic compositions.

### BACKGROUND OF THE DISCLOSURE

Many individuals suffer from dry and damaged hair. Dryness and damage can occur due to several factors including weather exposure, mechanical treatments (e.g. brushing hair), excessive treatments using chemicals, dying hair, heat styling, etc. In combination, using cleansing products that can be excessively stripping of hair's natural oils, can also lead to split ends, dull hair, and exacerbate dry hair. To mitigate the damage, many individuals utilize conditioners or other hair treatments for mitigating the damage and improve the health of hair.

Conditioners are typically emulsions having a dispersed oil phase and a continuous aqueous phase. As a result of logistic systems, cosmetic products must be formulated to withstand both high temperatures and low temperatures associated with transportation (e.g., in back of a truck) from the manufacturing plant to the warehouse storage to the retail stores and, ultimately, to the consumer. Accordingly, conditioners are typically formulated to maintain a stable emulsion over large temperature ranges as well as repeated temperature swings.

There is an ongoing need for new and improved formulations that offer consumers a unique sensorial experience. This unique sensorial experience may be related to product performance and/or product texture and consistency and/or tactile experience.

### SUMMARY OF THE DISCLOSURE

The present disclosure relates to cosmetic compositions that provide a unique sensorial and consumer experience. The cosmetic compositions may be heat and/or shear activated, such that the cosmetic compositions undergo a sensorial and/or tactile changes. For example, in some instances, the cosmetic compositions transition from exhibiting a more solid-like behavior to exhibiting a more liquid-like behavior in the presence of heat activation and/or shear, such as provided by a user rubbing the cosmetic composition between his or her hands or applying the cosmetic composition during a warm shower or bath. In some instances, the viscosity of the cosmetic composition may transition from a viscosity of about 10 to about 500 Pa·s at a temperature of 24° C. to a reduced viscosity of less than 100 Pa·s at a temperature of 40° C. as measured with LV 04 Spindle on a Brookfield DV2T viscometer at a range of 1 to 10 rpm after 90 seconds.

The cosmetic compositions typically include:
a) about 20 to about 84.5 wt. % of one or more glycols chosen from ethylene glycol, propylene glycol, butylene glycol, hexylene glycol, pentylene glycol, diethylene glycol, dipropylene glycol, 1,3 propanediol, polyethylene glycols, and a mixture thereof;
(b) about 15 wt. % to about 50 wt. % of one or more trihydric alcohols having from 3 to 5 carbon atoms;
   wherein the total amount of the one or more glycols of (a) is greater than the total amount of the one or more trihydric alcohols of (b);
(c) about 0.1 to about 5 wt. % of one or more cationic surfactants;
(d) about 0.1 to about 5 wt. % one or more alkoxylated fatty alcohols; and
(e) about 0.1 to about 10 wt. % of one or more fatty alcohols;
   wherein all percentages by weight are based on the total weight of the cosmetic composition.

The cosmetic composition is typically a substantially anhydrous composition and typically has a lamellar network. The weight ratio of the combined weight of the one or more cationic surfactants of (c) and the one or more alkoxylated fatty alcohols of (d) relative to the combined weight of the one or more cationic surfactants of (c), the one or more alkoxylated fatty alcohols of (d), and the one or more of fatty alcohols of (e) is from about 0.2:1 to about 0.5:1. The composition may transition from having a viscosity of about 10 to about 500 Pa·s at 24° C. to having a viscosity of less than 100 Pa·s at 40° C., as measured with LV 04 Spindle on a Brookfield DV2T viscometer at a range of 1 to 10 rpm after 90 seconds.

In at least one example of the cosmetic composition, the one or more glycols comprises propylene glycol. The one or more tryihdyric alcohols may be chosen from glycerol, a butanetriol, a pentanetriol, and a mixture thereof. For example, the one or more tryihdyric alcohols may be chosen from glycerol, 1,2,3-butanetriol, 1,2,4-butanetriol, 1,2,5-pentanetriol, and a mixture thereof. In one instance, the one or more tryihdyric alcohols includes or consists of glycerin.

The one or more cationic surfactants may, in some cases, be chosen from quaternary ammonium compounds, amidoamines, or a mixture thereof. Non-limiting examples of cationic surfactants include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

The cosmetic composition may also include one or more cationic polymers, such as polyquaternium-67. Additionally or alternatively, the fatty alcohol group of the one or more alkoxylated fatty alcohols may have a carbon chain of 8 to 50 carbons. In some cases, the one or more alkoxylated fatty alcohols are alkoxylated with about 1 to about 100 moles of an alkylene oxide per mole of alkoxylated fatty alcohol.

The one or more fatty alcohols may have a carbon chain of 12 to 22 carbons. Non-limiting examples of the one or more fatty alcohols include decyl alcohol, undecyl alcohol, dodecyl alcohol, myristyl alcohol, cetearyl alcohol, cetyl alcohol, stearyl alcohol, isostearyl alcohol, isocetyl alcohol, behenyl alcohol, linalool, oleyl alcohol, cis-4-t-butylcyclohexanol, isotridecyl alcohol, myricyl alcohol and a mixture thereof.

In some instances, the cosmetic composition is free of water. Additionally or alternatively, the cosmetic composition may be free of silicones.

The instant disclosure also relates to methods for treating hair. Exemplary methods for treating hair include applying the cosmetic compositions disclosed herein to hair and, optionally, rinsing the composition from the hair. In some instances, the method for treating the hair includes one or more of the following: mixing the cosmetic composition with shampoo prior to application to hair; layering the cosmetic composition onto hair with a shampoo; applying the cosmetic composition to hair after a shampoo has been rinsed from the hair; layering the cosmetic composition onto hair with a conditioner; mixing the cosmetic composition with a conditioner prior to application to hair; and/or applying the cosmetic composition to hair after a conditioner has been rinsed from the hair.

### BRIEF DESCRIPTION OF THE DRAWING

Implementation of the present technology will now be described, by way of example only, with reference to the attached figures, wherein:

FIG. 1 is a graph of the storage modulus and the loss modulus of a traditional conditioner and exemplary compositions according to aspects of the disclosure during a temperature sweep experiment; and

FIG. 2 includes pictures of hair swatches that were treated with comparative compositions or exemplary compositions according to aspects of the disclosure.

It should be understood that the various aspects are not limited to the arrangements and instrumentality shown in the drawings.

### DETAILED DESCRIPTION OF THE DISCLOSURE

The cosmetic compositions of the instant disclosure provide a unique sensorial experience. The cosmetic composition may be heat and/or shear activated, such that the cosmetic composition undergoes a sensorial and/or tactile change during use (e.g., by a user rubbing the cosmetic composition between their hands or on hair). For example, the cosmetic composition may transition from exhibiting more solid-like behavior to exhibiting more liquid-like behavior upon being heat and/or shear activated.

The cosmetic composition may have a viscosity of about 10 to about 500 Pa·s at a temperature of 24° C. and may transition to having a viscosity of less than 100 Pa·s at a temperature of 40° C., as measured with LV 04 Spindle on a Brookfield DV2T viscometer at a range of 1 to 10 rpm after 90 seconds. For instance, the viscosity of the cosmetic composition at a temperature of 24° C. may range from about 10 to about 500 Pa·s, about 20 to about 400 Pa·s, about 30 to about 300 Pa·s, about 40 to about 200 Pa·s, or about 50 to about 150 Pa·s.

After transitioning to a composition exhibiting more liquid-like behavior and/or having a temperature of 40° C., the cosmetic composition may have a reduced viscosity, as measured with LV 04 Spindle on a Brookfield DV2T viscometer at a range of 1 to 10 rpm after 90 seconds, such as less than 100 Pa·s, less than 95 Pa·s, less than 90 Pa·s, less than 85 Pa·s, less than 80 Pa·s, less than 75 Pa·s, less than 70 Pa·s, less than 65 Pa·s, less than 60 Pa·s, less than 55 Pa·s, less than 50 Pa·s, less than 45 Pa·s, less than 40 Pa·s, less than 35 Pa·s, less than 30 Pa·s, less than 25 Pa·s, less than 20 Pa·s, less than 15 Pa·s, or less than 10 Pa·s. Preferably, the viscosity of the cosmetic composition is lower at a temperature of 40° C. than at a temperature of 24° C., as measured with LV 04 Spindle on a Brookfield DV2T viscometer at a range of 1 to 10 rpm after 90 seconds.

Additionally and/or alternatively, the cosmetic composition may transition from a viscoelastic solid to a viscoelastic liquid upon heat and/or shear activation. As used herein, a viscoelastic solid composition has a storage modulus (G) value that is greater than its loss modulus (G″) value at a stress of 0.5 Pa and a frequency of 3 Hz. A viscoelastic liquid composition has a storage modulus (G) value that is less than its loss modulus (G″) value at a stress of 0.5 Pa and a frequency of 3 Hz. The storage modulus (G′) and loss modulus (G″) may be determined at a stress of 0.5 Pa and a frequency of 3 Hz using an AR G2 Rheometer in conjunction with a Smart Swap™ Peltier plate temperature system.

The storage modulus (G′) describes the level of solid-state behavior exhibited by the cosmetic composition. The loss modulus (G″) describes the level of liquid-state behavior exhibited by the cosmetic composition. Thus, the level of the storage modulus to the level of the loss modulus affects the sensational experience and tactile perception of the cosmetic composition. For example, the cosmetic composition may transition from a viscoelastic solid composition to a viscoelastic liquid composition having a loss modulus that is greater than the storage modulus by at least 5%, at least 10%, at least 13%, at least 16%, at least 19%, at least 23%, at least 26%, or at least 29% at a temperature of 50° C. or less, preferably 45° C. or less, preferably 40° C. or less, preferably 38° C. or less, or preferably 35° C. or less. In some instances, the cosmetic composition transitions from a viscoelastic solid composition to a viscoelastic liquid composition at a temperature of a typical shower (˜30 to ˜50° C.), e.g., such that the transition occurs as the user applies the cosmetic composition to the hair and/or scalp and/or body and/or occurs in the hands of the user as he or she rubs it in his or her hands.

The cosmetic compositions typically have a lamellar network and include:
a) about 20 to about 84.5 wt. % of one or more glycols chosen from ethylene glycol, propylene glycol, butylene glycol, hexylene glycol, pentylene glycol, diethylene glycol, dipropylene glycol, 1,3 propanediol, polyethylene glycols, and a mixture thereof;
(b) about 15 wt. % to about 50 wt. % of one or more trihydric alcohols having from 3 to 5 carbon atoms;
   wherein the total amount of the one or more glycols of (a) is greater than the total amount of the one or more trihydric alcohols of (b);
(c) about 0.1 to about 5 wt. % of one or more cationic surfactants;
(d) about 0.1 to about 5 wt. % one or more alkoxylated fatty alcohols; and
(e) about 0.1 to about 10 wt. % of one or more fatty alcohols,
   wherein all percentages by weight are based on the total weight of the cosmetic composition.

The cosmetic composition may be translucent and, optionally, include one or more colorants and/or pigments. As used herein, the term “translucent” refers to the characteristic of a composition to permit the passage of light but does not necessarily allow for detailed objects to be distinguished through a thickness of 1 cm of the cosmetic composition. Although the cosmetic composition may be translucent, the cosmetic compositions may optionally include colorants and/or pigments that provide a color to the cosmetic composition while also being translucent.

In some instances, the cosmetic composition is free or substantially free of water (anhydrous or substantially anhydrous). Alternatively or additionally, the composition may have an amount of water that is less than 6 wt. %, preferably less than 5 wt. %, preferably less than 4 wt. %, preferably less than 3 wt. %, preferably less than 2 wt. %, preferably less than 1 wt. %, and/or less than 0.5 wt. %, based on the total weight of the cosmetic composition.

The cosmetic composition may be free of or substantially free of monoalcohols having from 1 to 8 carbons. In some instances, the composition may have an amount of monoalcohols having from 1 to 8 carbons that is less than 8 wt. %, preferably less than 7 wt. %, preferably less than 6 wt. %, preferably less than 5 wt. %, preferably less than 4 wt. %, preferably less than 3 wt. %, preferably less than 2 wt. %, preferably less than 1 wt. %, preferably less than 0.5 wt. %, and/or preferably less than 0.1 wt. %, based on the total weight of the cosmetic composition.

In some instances, the cosmetic composition is free or substantially free of silicone. In some instances, the composition may have an amount of silicone that is less than 6 wt. %, preferably less than 5 wt. %, preferably less than 4 wt. %, preferably less than 3 wt. %, preferably less than 2 wt. %, preferably less than 1 wt. %, preferably less than 0.5 wt. %, and/or preferably less than 0.1 wt. %, based on the total weight of the cosmetic composition.

The cosmetic composition may be free of or substantially free of polyurethane resin. In some cases, the composition may have an amount of polyurethane resin that is less than 6 wt. %, preferably less than 5 wt. %, preferably less than 4 wt. %, preferably less than 3 wt. %, preferably less than 2 wt. %, preferably less than 1 wt. %, preferably less than 0.5 wt. %, and/or preferably less than 0.1 wt. %, based on the total weight of the cosmetic composition.

Additionally or alternatively, the cosmetic composition may be free of or substantially free of thickening agents. In some instances, the composition may have an amount of thickening agents that is less than 6 wt. %, preferably less than 5 wt. %, preferably less than 4 wt. %, preferably less than 3 wt. %, preferably less than 2 wt. %, preferably less than 1 wt. %, preferably less than 0.5 wt. %, and/or preferably less than 0.1 wt. %, based on the total weight of the cosmetic composition.

Glycol(s)

The cosmetic compositions typically include one or more glycols chosen from ethylene glycol, propylene glycol, butylene glycol, hexylene glycol, pentylene glycol, diethylene glycol, dipropylene glycol, 1,3 propanediol, polyethylene glycols, and a mixture thereof. The amount of glycols is typically in an amount of about 20 to about 84.5 wt. %. In some instances, the amount of glycols in the cosmetic composition may be present in amount of about 20 to about 84.5 wt. %, about 30 to about 84.5 wt. %, about 40 to about 84.5 wt. %, about 50 to about 84.5 wt. %, about 55 to about 84.5 wt. %, about 60 to about 84.5 wt. %, about 65 to about 84.5 wt. %; about 20 to about 80 wt. %, about 30 to about 80 wt. %, about 40 to about 80 wt. %, about 50 to about 80 wt. %, about 55 to about 80 wt. %, about 60 to about 80 wt. %, about 65 to about 80 wt. %; about 20 to about 75 wt. %, about 30 to about 75 wt. %, about 40 to about 75 wt. %, about 50 to about 75 wt. %, about 55 to about 75 wt. %, about 60 to about 75 wt. %, about 65 to about 75 wt. %; about 20 to about 70 wt. %, about 30 to about 70 wt. %, about 40 to about 70 wt. %, about 50 to about 70 wt. %, about 55 to about 70 wt. %, about 60 to about 70 wt. %, or about 65 to about 70 wt. %, including all ranges and sub-ranges therebetween, based on the total weight of the cosmetic composition.

The one or more glycols may include primarily (or exclusively) propylene glycol. For example, propylene glycol may comprise at least 50 wt. %, at least 60 wt. %, at least 70 wt. %, at least 80 wt. %, at least 90 wt. %, or at least 95 wt. % of the one or more glycols, based on the total weight of the one or more glycols in the cosmetic composition. In at least one instance, the one or more glycols is exclusively propylene glycol.

Trihydric Alcohol(s)

The cosmetic compositions include one or more trihydric alcohols having from 3 to 5 carbon atoms typically in an amount of about 15 wt. % to about 50 wt. %. For example, the amount of trihydric alcohols having from 3 to 5 carbon atoms present in the cosmetic composition may be from about 15 wt. % to about 50 wt. %, about 18 wt. % to about 50 wt. %, about 20 wt. % to about 50 wt. %, about 23 wt. % to about 50 wt. % about 25 wt. % to about 50 wt. %, about 30 wt. % to about 50 wt. %, about 35 wt. % to about 50 wt. %, about 40 wt. % to about 50 wt. %; about 15 wt. % to about 45 wt. %, about 18 wt. % to about 45 wt. %, about 20 wt. % to about 45 wt. %, about 23 wt. % to about 45 wt. % about 25 wt. % to about 45 wt. %, about 30 wt. % to about 45 wt. %, about 35 wt. % to about 45 wt. %, about 40 wt. % to about 45 wt. %; about 15 wt. % to about 40 wt. %, about 18 wt. % to about 40 wt. %, about 20 wt. % to about 40 wt. %, about 23 wt. % to about 40 wt. % about 25 wt. % to about 40 wt. %, about 30 wt. % to about 40 wt. %, or about 35 wt. % to about 40 wt. %, including all ranges and sub-ranges therebetween, based on the total weight of the cosmetic composition.

The cosmetic compositions typically include a total amount of the one or more glycols that is greater than the total amount of the one or more trihydric alcohols. For example, the amount of glycols may be greater than the amount of trihydric alcohols having from 3 to 5 carbon atoms by, e.g., a multiple of at least 1.01, at least 1.03, at least 1.05, at least 1.08, at least 1.1, at least 1.15, at least 1.2, at least 1.3, at least 1.4, at least 1.5, at least 1.75, at least 2, at least 2.5, at least 3, at least 3.5, at least 4, at least 4.5, at least 5, at least 5.5, at least 6, or at least 6.5. In at least one case, the total amount of the one or more glycols is preferably a multiple of about 4.5 to about 5.5 greater than the total amount of the one or more trihydric alcohols.

The one or more tryihdyric alcohols having from 3 to 5 carbon atoms may include or be chosen from glycerol, a butanetriol, a pentanetriol, and a mixture thereof. Non-limiting examples of tryihdyric alcohols that may be suitable include glycerol, 1,2,3-butanetriol, 1,2,4-butanetriol, 1,2,5-pentanetriol, and a mixture thereof. In some instances, the one or more tryihdyric alcohols having from 3 to 5 carbon atoms include glycerin. Glycerin may comprise the majority of the amount of tryihdyric alcohols having from 3 to 5 carbon atoms. For example, the glycerin may comprise at least 50 wt. %, at least 60 wt. %, at least 70 wt. %, at least 80 wt. %, at least 90 wt. %, or at least 95 wt. % of the one or more tryihdyric alcohols, based on the total weight of the one or more tryihdyric alcohols having from 3 to 5 carbon atoms in the cosmetic composition. In at least one instance, the one or more tryihdyric alcohols is exclusively glycerin.

Cationic Surfactant(s)

The cosmetic composition includes one or more cationic surfactant(s) typically in an amount ranging from about 0.1 to about 5 wt. % of, based on the total weight of the cosmetic composition. For example, the amount of cationic surfactants present in the cosmetic composition may range from about 0.1 to about 5 wt. %, about 0.1 to about 4 wt. %, about 0.1 to about 3 wt. %, about 0.1 to about 2 wt. %; about 0.5 to about 5 wt. %, about 0.5 to about 4 wt. %, about 0.5 to about 3 wt. %, about 0.5 to about 2 wt. %; about 1 to about 5 wt. %, about 1 to about 4 wt. %, about 1 to about 3 wt. %, or about 1 to about 2 wt. %, including all ranges and sub-ranges therebetween, based on the total weight of the cosmetic composition.

The term “cationic surfactant” means a surfactant that may be positively charged when it is contained in the cosmetic compositions according to the disclosure. This surfactant may bear one or more positive permanent charges or may contain one or more functional groups that are cationizable in the composition according to the disclosure.

The one or more cationic surfactants include or are chosen from quaternary ammonium compounds, amidoamines, or a mixture thereof. Examples of cationic surfactants that may be suitable for the cosmetic composition include or may be chosen from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

Additional, non-limiting examples of cationic surfactants include: cetyl trimethyl ammonium chloride available, for example, with trade name CA-2350 from Nikko Chemicals and CTAC 30KC available from KCl, stearyl trimethyl ammonium chloride with trade name Arquad 18/50 available from Akzo Nobel, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methy) pyridinium chloride.

Hydrophilically substituted cationic surfactants in which at least one of the substituents contain one or more aromatic, ether, ester, amido, or amino moieties present as substituents or as linkages in the chain may also be in the cosmetic composition. Non-limiting examples of hydrophilically substituted cationic surfactants that may be useful in the cosmetic compositions include the materials having the following INCI designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and mixtures thereof.

In one embodiment, the hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, alkyl amidopropyl trimonium salt, polyoxyethylene alkyl ammonium salt, and mixtures thereof. For example, commercially available hydrophilically substituted cationic surfactants may include those under the following trade names; VARISOFT 110, VARISOFT PATC, VARIQUAT K1215 and 638 from Witco Chemical, ETHOQUAD 18/25, ETHOQUAD O/12PG, ETHOQUAD C/25, and ETHOQUAD S/25 from Akzo, DEHYQUART SP from Cognis, and MONAQUAT ISEIS, and MONAQUAT SL-5 available from Uniqema.

In some embodiments, the cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

In some embodiments, the cationic surfactant is selected from cetrimonium chloride, behentrimonium chloride, and mixtures thereof.

Non-polymeric, mono-, di-, and/or tri-carboxylic acids may be used to “neutralize” the amidoamines. In some cases, the one or more non-polymeric, mono-, di-, and/or tri-carboxylic acids include at least one dicarboxylic acid. Non-limiting examples include lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, and mixtures thereof. In particular, lactic acid or tartaric acid or mixtures thereof are useful, especially in combination with amidoamines such as, for example, stearamidopropyl dimethylamine

Alkoxylated Fatty Alcohol(s)

The cosmetic compositions include one or more alkoxylated fatty alcohols typically in an amount of about 0.1 to about 5 wt. %, based on the total weight of the cosmetic composition. For example, the amount of alkoxylated fatty alcohols present in the cosmetic composition may range from about 0.1 to about 5 wt. %, about 0.1 to about 4 wt. %, about 0.1 to about 3 wt. %, about 0.1 to about 2 wt. %.%, about 0.1 to about 1.5 wt. %, about 0.1 to about 1 wt. %; about 0.5 to about 5 wt. %, about 0.5 to about 4 wt. %, about 0.5 to about 3 wt. %, about 0.5 to about 2 wt. %, about 0.5 to about 1.5 wt. %, about 0.5 to about 1 wt. %; about 1 to about 5 wt. %, about 1 to about 4 wt. %, about 1 to about 3 wt. %, or about 1 to about 2 wt. %, including all ranges and sub-ranges therebetween, based on the total weight of the cosmetic composition.

The alkoxylated fatty alcohols may be alkoxylated with about 1 to about 100 moles of an alkylene oxide per mole of alkoxylated fatty alcohol. For example, the alkoxylated fatty alcohols may be alkoxylated with about 1 to about 80 moles, about 2 to about 50, about 5 to about 45 moles, about 10 to about 40 moles, or 15 to about 35 mores, including all ranges and subranges therebetween, of an alkylene oxide per mole of alkoxylated fatty alcohol.

Additionally or alternatively, the alkoxylated fatty alcohols may have a fatty alcohol group that has a carbon chain of greater than 8 carbon atoms, or 8 to 50 carbon atoms, or 8 to 40 carbon atoms, or 8 to 30 carbon atoms, or 8 to 22 carbon atoms, or 12 to 22 carbon atoms, or 12 to 18 carbon atoms, including all ranges and subranges therebetween. In some instances, the fatty alcohol group of the alkoxylated fatty alcohols has a carbon chain of 10 to 20 carbon atoms or 10 to 18 carbon atoms. The alkoxylated fatty alcohols may be chosen from polyethylene glycol ethers, such as those having a fatty alcohol group with a carbon chain of 12 to 16 or 12 to 14 carbon atoms. The fatty alcohol portion is preferably hydrogenated (for example, stearyl, lauryl, cetyl, cetearyl); however, the fatty alcohol may contain one or more double bonds (for example, oleyl).

As examples of alkoxylated fatty alcohols, steareth (for example, steareth-2, steareth-20, and steareth-21), laureth (for example, laureth-4, and laureth-12), ceteth (for example, ceteth-10 and ceteth-20) and ceteareth (for example, ceteareth-2, ceteareth-10, and ceteareth-20) are mentioned.

In at least one instance, the one or more alkoxylated fatty alcohols include steareth-20. In some instances, the one or more alkoxylated fatty alcohols may be exclusively steareth-20.

Fatty Alcohol(s)

The cosmetic compositions include one or more fatty alcohols in addition to the alkoxylated fatty alcohols discussed above typically in an amount of about 0.1 to about 10 wt. %, based on the total weight of the cosmetic composition. For example, the amount of fatty alcohols, other than alkoxylated fatty alcohols, in the cosmetic composition may range from about 0.1 to about 10 wt. %, about 0.1 to about 9 wt. %, about 0.1 to about 8 wt. %, about 0.1 to about 7 wt. %, about 0.1 to about 6 wt. %, about 0.1 to about 5 wt. %, about 0.1 to about 4 wt. %, about 0.1 to about 3 wt. %, about 0.1 to about 2 wt. %; about 0.5 to about 10 wt. %, about 0.5 to about 9 wt. %, about 0.5 to about 8 wt. %, about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %; about 1 to about 10 wt. %, about 1 to about 9 wt. %, about 1 to about 8 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; about 1.5 to about 5 wt. %, about 1.5 to about 4 wt. %, about 1.5 to about 3 wt. %, or about 1.5 to about 2.5 wt. %, including all ranges and sub-ranges therebetween, based on the total weight of the cosmetic composition.

Suitable fatty alcohols include those having from about 8 to about 30 carbon atoms, from about 12 to about 22 carbon atoms, and from about 14 to about 22 carbon atoms. These fatty alcohols can be straight or branched chain alcohols and can be saturated or unsaturated. Non-limiting examples of fatty alcohols include decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, and a mixture thereof. In some cases, the fatty alcohols comprise at least one of or may be chosen from myristyl alcohol, lauryl alcohol, cetyl alcohol, stearyl alcohol, cetearyl alcohol, isostearyl alcohol, oleyl alcohol, isotridecyl alcohol, and a mixture thereof.

The fatty alcohol may be saturated or unsaturated. Exemplary saturated liquid fatty alcohols may be branched and optionally contain in their structure at least one aromatic or non-aromatic ring. In some instances, however, the fatty alcohols are acyclic. Non-limiting examples of liquid saturated fatty alcohols include octyldodecanol, isostearyl alcohol, and 2-hexyldecanol.

Exemplary unsaturated liquid fatty alcohol may include in their structure at least one double or triple bond. For example, the fatty alcohols may include several double bonds (such as 2 or 3 double bond), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol and undecylenyl alcohol.

Cationic Polymers

The cosmetic compositions may include one or more cationic polymers. The cationic polymers can comprise mixtures of monomer units derived from amine- and/or quaternary ammonium-substituted monomer and/or compatible spacer monomers.

Suitable cationic polymers include, for example: copolymers of 1-vinyl-2-pyrrolidine and 1-vinyl-3-methyl-imidazolium salt (e.g., chloride salt) (referred to as Polyquaternium-16) such as those commercially available from BASF under the LUVIQUAT tradename (e.g., LUVIQUAT FC 370); copolymers of 1-vinyl-2-pyrrolidine and dimethylaminoethyl methacrylate (referred to as Polyquaternium-11) such as those commercially from Gar Corporation (Wayne, N.J., USA) under the GAFQUAT tradename (e.g., GAFQUAT 755N); and cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7).

Other cationic polymers that may be used include polysaccharide polymers, such as cationic cellulose derivatives and cationic starch derivatives. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). These materials are available from Amerchol Corp. (Edison, N.J., USA) under the tradename Polymer LM-200. Additionally or alternatively, the cationic conditioning polymers may include or be chosen from cationic guar gum derivatives, such as guar hydroxypropyltrimonium chloride.

The cosmetic composition may include or be chosen from polyquaterniums. For example, the cosmetic composition may include Polyquaternium-1 (ethanol, 2,2′,2″-nitrilotris-, polymer with 1,4-dichloro-2-butene and N,N,N′,N′-tetramethyl-2-butene-1,4-diamine), Polyquaternium-2, (poly[bis(2-chloroethyl) ether-alt-1,3-bis[3-(dimethylamino)propyl]urea]), Polyquaternium-4, (hydroxyethyl cellulose dimethyl diallylammonium chloride copolymer; Diallyldimethylammonium chloride-hydroxyethyl cellulose copolymer), Polyquaternium-5 (copolymer of acrylamide and quaternized dimethylammoniumethyl methacrylate), Polyquaternium-6 (poly(diallyldimethylammonium chloride)), Polyquaternium-7 (copolymer of acrylamide and diallyldimethylammonium chloride), Polyquaternium-8 (copolymer of methyl and stearyl dimethylaminoethyl ester of methacrylic acid, quaternized with dimethylsulphate), Polyquaternium-9 (homopolymer of N,N-(dimethylamino)ethyl ester of methacrylic acid, quaternized with bromomethane), Polyquaternium-10 (quaternized hydroxyethyl cellulose), Polyquaternium-11 (copolymer of vinylpyrrolidone and quaternized dimethylaminoethyl methacrylate), Polyquaternium-12 (ethyl methacrylate/abietyl methacrylate/diethylaminoethyl methacrylate copolymer quaternized with dimethyl sulfate), Polyquaternium-13 (ethyl methacrylate/oleyl methacrylate/diethylaminoethyl methacrylate copolymer quaternized with dimethyl sulfate), Polyquaternium-14 (trimethylaminoethylmethacrylate homopolymer), Polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), Polyquaternium-19 (copolymer of polyvinyl alcohol and 2,3-epoxypropylamine), Polyquaternium-20 (copolymer of polyvinyl octadecyl ether and 2,3-epoxypropylamine), Polyquaternium-22 (copolymer of acrylic acid and diallyldimethylammonium chloride), Polyquaternium-24 (auaternary ammonium salt of hydroxyethyl cellulose reacted with a lauryl dimethyl ammonium substituted epoxide), Polyquaternium-27 (block copolymer of Polyquaternium-2 and Polyquaternium-17), Polyquaternium-28 (copolymer of vinylpyrrolidone and methacrylamidopropyl trimethylammonium), Polyquaternium-29 (chitosan modified with propylen oxide and quaternized with epichlorhydrin), Polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), Polyquaternium-31 (N,N-dimethylaminopropyl-N-acrylamidine quatemized with diethylsulfate bound to a block of polyacrylonitrile), Polyquaternium-32 (poly(acrylamide 2-methacryloxyethyltrimethyl ammonium chloride)), Polyquaternium-33 (copolymer of trimethylaminoethylacrylate salt and acrylamide), Polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), Polyquaternium-36 (copolymer of N,N-dimethylaminoethylmethacrylate and buthylmethacrylate, quaternized with dimethylsulphate), Polyquaternium-37 (poly(2-methacryloxyethyltrimethylammonium chloride)), Polyquaternium-39 (terpolymer of acrylic acid, acrylamide and diallyldimethylammonium Chloride), Polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (copolymer of (N-methyl-N-ethoxyglycine)methacrylate and N,N-dimethylaminoethylmethacrylate, quaternized with dimethyl sulphate), Polyquaternium-46 (terpolymer of vinylcaprolactam, vinylpyrrolidone, and quaternized vinylimidazole), Polyquaternium-47 (terpolymer of acrylic acid, methacrylamidopropyl trimethylammonium chloride, and methyl acrylate), and/or Polyquaternium-67.

In some instances, the cosmetic compositions of the instant disclosure include one or more cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful.

The amount of cationic polymers in the cosmetic composition typically ranges from about 0.01 to about 20 wt. % of the total weight of the cosmetic composition. In some instances, the conditioning agents are in an amount ranging from about 0.01 to about 20 wt. %, about 0.01 to about 15 wt. %, about 0.01 to about 10 wt. %, about 0.01 to about 8 wt. %, about 0.01 to about 6 wt. %; about 0.1 to about 20 wt. %, about 0.1 to about 15 wt. %, about 0.1 to about 10 wt. %, about 0.1 to about 8 wt. %, about 0.1 to about 6 wt. %; from about 0.5 to about 25 wt. % about 0.5 to about 20 wt. %, about 0.5 to about 15 wt. %, about 0.5 to about 10 wt. %, about 0.5 to about 8 wt. %, about 0.5 to 6 wt. %; from about 1 to about 25 wt. %, about 1 to about 20 wt. %, about 1 to about 15 wt. %, about 1 to about 10 wt. %, about 1 to about 8 wt. %, about 1 to about 6 wt. %; from about 1.5 to about 25 wt. %, about 1.5 to about 20 wt. %, about 1.5 to about 15 wt. %, about 1.5 to about 10 wt. %, about 1.5 to about 8 wt. %, or about 1.5 to about 6 wt. %, including ranges and sub-ranges therebetween, based on the total weight of the cosmetic composition.

Nonionic Surfactant(s)

The cosmetic compositions may, optionally, include one or more nonionic surfactants in addition to the alkoxylated fatty alcohols discussed above. The amount of nonionic surfactants, if present, typically range from about 0.05 to about 6 wt. % of the total weight of the cosmetic composition. For example, the total weight of the plurality of nonionic surfactants may range from about 0.05 to about 6 wt. %, 0.05 to about 5 wt. %, 0.05 to about 4 wt. %, 0.05 to about 3 wt. %; from 0.1 to about 6 wt. %, 0.1 to about 5 wt. %, 0.1 to about 4 wt. %, 0.1 to about 3 wt. %; from 0.5 to about 6 wt. %, 0.5 to about 5 wt. %, 0.5 to about 4 wt. %, 0.5 to about 3 wt. %; from 0.8 to about 6 wt. %, 0.8 to about 5 wt. %, 0.8 to about 4 wt. %, 0.8 to about 3 wt. %; from 1 to about 6 wt. %, 1 to about 5 wt. %, 1 to about 4 wt. %, or 1 to about 3 wt. %, including ranges and sub-ranges therebetween, based on the total weight of the cosmetic composition.

Examples of nonionic surfactants that may, in some cases, be suitably incorporated into the cosmetic composition include and/or may be chosen from alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures thereof.

As glyceryl esters of fatty acids, glyceryl stearate (glyceryl mono-, di- and/or tristearate) (INCI name: glyceryl stearate) or glyceryl ricinoleate and mixtures thereof can be cited. As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Additionally or alternatively, the nonionic surfactants may comprise or be selected from alkanolamides, polyglucosides, sorbitan derivatives, and polyol esters.

Alkanolamide(s)

Non-limiting examples alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one −OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides).

Suitable fatty acid alkanolamides may include those formed by reacting an alkanolamine and a C6-C36 fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof.

In some instances, the fatty acid alkanolamides preferably include cocamide MIPA, cocamide DEA, cocamide MEA, cocamide DIPA, and mixtures thereof. In particular, the fatty acid alkanolamide may be cocamide MIPA, which is commercially available under the tradename EMPILAN from Innospec Active Chemicals.

Fatty acid alkanolamides include those of the following structure:

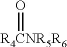

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and mixtures thereof); wherein R₅ is selected from —CH₂OH, —CH₂CH₂OH, —CH₂CH₂CH₂OH, —CH₂(CHOH)₄CH₂OH, -benzyl, and mixtures thereof; and wherein R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof.

In some instances, the one or more of the fatty acid alkanolamides include one or more acyl glucamides, for example, acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide

Alkyl Polyglucoside(s)

In some embodiments, the one or more alkyl polyglucosides include those chosen from lauryl glucoside, octyl glucoside, decyl glucoside, coco glucoside, caprylyl/capryl glucoside, sodium lauryl glucose carboxylate, and a mixture thereof. In some cases, the alkyl polyglucosides includes or is chosen from lauryl glucoside. Additionally or alternatively, the alkyl polyglucosides may be chosen from glycerol (C₆-C₂₄)alkylpolyglycosides including, e.g., polyethoxylated fatty acid mono or diesters of glycerol (C₆-C₂₄)alkylpolyglycosides. Additional alkyl polyglucosides that may be suitably incorporated, in some instances, in the cosmetic composition includes alkyl polyglucosides having a structure according to the following formula:

R¹—O—(R²O)ₙ—Z(x)

wherein R¹ is an alkyl group having 8-18 carbon atoms;

R² is an ethylene or propylene group;

Z is a saccharide group with 5 to 6 carbon atoms;

n is an integer from 0 to 10; and

x is an integer from 1 to 5.

Useful alkyl poly glucosides may, in some instances, include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

Sorbitan Derivative(s)

Suitable sorbitan derivatives that may be incorporated into the plurality of nonionic surfactants include those chosen from polysorbate-20 (POE(20) sorbitan monolaurate), polysorbate-21 (POE(4) sorbitan monolaurate), polysorbate-40 (POE(20) sorbitan monopalmitate), polysorbate-60 (POE(20) sorbitan monostearate), polysorbate-61 (POE(4) sorbitan monostearate), polysorbate-65 (POE(20) sorbitan tristearate), polysorbate-80 (POE(20)sorbitan monooleate), polysorbate-81 (POE(4) sorbitan monooleate), polysorbate 85 (POE(20) Sorbitan Trioleate), sorbitan isostearate, sorbitan monolaurate, sorbitan monooleate, sorbitan monopalmitate, sorbitan monostearate, sorbitan sesquioleate, sorbitan trioleate and sorbitan tristearateand a mixture thereof.

Additional and/or alternative sorbitan derivatives include sorbitan esters including, e.g., esters of C₁₆-C₂₂ fatty acid and of sorbitan that were formed by esterification, with sorbitol, of at least one fatty acid comprising at least one saturated or unsaturated linear alkyl chain respectively having from 16 to 22 carbon atoms. These esters can be chosen in particular from sorbitan stearates, behenates, arachidates, palmitates or oleates, and their mixtures. Examples of optional sorbitan esters include sorbitan monostearate (INCI name:Sorbitan stearate) sold by Croda under the name Span 60, the sorbitan tristearate sold by Croda under the name Span 65 V, the sorbitan monopalmitate (INCI name:Sorbitan palmitate) sold by Croda under the name Span 40, the sorbitan monooleate sold by Croda under the name Span 80 V or the sorbitan trioleate sold by Uniqema under the name Span 85 V. Preferably, the sorbitan ester used is sorbitan tristearate.

Polyol Ester(s)

Non-limiting examples of the one or more polyol esters include those chosen from alkoxylated polyol esters. For instance, the alkoxylated polyol esters may be chosen from pegylated derivatives of propylene glycol oleate, propylene glycol caprylate/caprate, propylene glycol cocoate, propylene glycol stearate, and a mixture thereof. In certain embodiments, the alkoxylated polyol esters are chosen from PEG-55 propylene glycol oleate, PEG-6 propylene glycol caprylate/caprate, PEG-8 propylene glycol cocoate, PEG-25 propylene glycol stearate, and PEG-120 propylene glycol stearate, and a mixture thereof. In some instances, the polyol ester is or includes PEG-55 propylene glycol oleate. Additionally and/or alternatively, the polyol esters may be chosen from ethoxylated fatty acid esters of sorbitan comprising from 2 to 30 mol of ethylene oxide.

In some cases, the polyol ester may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the INCI names: PEG-9 laurate to PEG-50 laurate); PEG-9 to PEG-50 palmitate (as the INCI names: PEG-9 palmitate to PEG-50 palmitate); PEG-9 to PEG-50 stearate (as the INCI names: PEG-9 stearate to PEG-50 stearate); PEG-9 to PEG-50 palmitostearate; PEG-9 to PEG-50 behenate (as the INCI names: PEG-9 behenate to PEG-50 behenate); polyethylene glycol 100 EO monostearate (INCI name: PEG-100 stearate); and mixtures thereof.

Sources of unsaturated polyol esters of glycerol include synthesized oils, natural oils (e.g., vegetable oils, algae oils, bacterial derived oils, and animal fats), combinations of these, and the like. Non-limiting examples of vegetable oils include Abyssinian oil, Almond oil, Apricot oil, Apricot Kernel oil, Argan oil, Avocado oil, Babassu oil, Baobab oil, Black Cumin oil, Black Currant oil, Borage oil, Camelina oil, Carinata oil, Canola oil, Castor oil, Cherry Kernel oil, Coconut oil, Corn oil, Cottonseed oil, Echium oil, Evening Primrose oil, Flax Seed oil, Grape Seed oil, Grapefruit Seed oil, Hazelnut oil, Hemp Seed oil, Jatropha oil, Jojoba oil, Kukui Nut oil, Linseed oil, Macadamia Nut oil, Meadowfoam Seed oil, Moringa oil, Neem oil, Olive oil, Palm oil, Palm Kernel oil, Peach Kernel oil, Peanut oil, Pecan oil, Pennycress oil, Perilla Seed oil, Pistachio oil, Pomegranate Seed oil, Pongamia oil, Pumpkin Seed oil, Raspberry oil, Red Palm Olein, Rice Bran oil, Rosehip oil, Safflower oil, Seabuckthorn Fruit oil, Sesame Seed oil, Shea Olein, Sunflower oil, Soybean oil, Tonka Bean oil, Tung oil, Walnut oil, Wheat Germ oil, High Oleoyl Soybean oil, High Oleoyl Sunflower oil, High Oleoyl Safflower oil, High Erucic Acid Rapeseed oil, combinations of these, and the like. Non-limiting examples of animal fats include lard, tallow, chicken fat, yellow grease, fish oil, emu oil, combinations of these, and the like. Non-limiting example of a synthesized oil includes tall oil, which is a byproduct of wood pulp manufacture. In some embodiments, the natural oil is refined, bleached, and/or deodorized.

The polyol esters may optionally be a natural polyol esters chosen from vegetable oil, an animal fat, an algae oil and mixtures thereof; and said synthetic polyol ester is derived from a material selected from the group consisting of ethylene glycol, propylene glycol, glycerol, polyglycerol, polyethylene glycol, polypropylene glycol, poly(tetramethylene ether) glycol, pentaerythritol, dipentaerythritol, tripentaerythritol, trimethylolpropane, neopentyl glycol, a sugar, in one aspect, sucrose, and mixtures thereof.

Additional non-limiting examples of nonionic surfactants that may optionally be used in the cosmetic composition include and/or may be chosen from alkanolamides; polyoxyalkylenated nonionic surfactants; polyglycerolated nonionic surfactants; ethoxylated fatty esters; alcohols, alpha-diols, alkylphenols and esters of fatty acids, being ethoxylated, propoxylated or glycerolated; copolymers of ethylene oxide and/or of propylene oxide; condensates of ethylene oxide and/or of propylene oxide with fatty alcohols; polyethoxylated fatty amides; ethoxylated oils from plant origin; fatty acid esters of sucrose; fatty acid esters of polyethylene glycol;N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures thereof.

The cosmetic compositions of the instant disclosure may be incorporated into a kit. For example, kits may include at least one cosmetic composition according to the instant disclosure and one or more additional compositions, such as a shampoo, a conditioner, etc. The various compositions are separately contained in the kits. In some instances, the kits include one or more cosmetic compositions according the instant disclosure, a shampoo, a conditioner, a mask, and/or other hair treatment products, all of which are separately contained. Instructions, mixing components, brushes, gloves, measuring tools, etc., may also optionally be included in the kits.

The compositions of the kit may be packaged in a variety of different containers, such as, for example, a ready-to-use container. Non-limiting examples of useful packaging include tubes, jars, caps, unit dose packages, and bottles, including squeezable tubes, bottles, and sprayable containers. The packaging may be configured so that it can be attached to a wall, such as a wall in a bathroom, including walls of a shower or tub. For example, the packaging can be a container that is configured to attach to a wall, such that when pressure is applied to the container, the composition contained therein is expelled from one or more openings in the container. In some cases, the packaging is a tube, such as a tube with two compartments, or dual tubes, each forming a separate compartment. Each compartment may include a different composition. For example, one tube or compartment may include a cosmetic composition according to the instant disclosure, and the other tube may include a composition to be used with the cosmetic composition, for example, a shampoo, a conditioner, an all-in-one shampoo/conditioner (i.e., a conditioning shampoo; also referred to as a “co-wash”) mask or other hair treatment products.

### Methods of Treating Hair

Methods of treating hair according to the disclosure may vary but typically include applying a cosmetic composition as disclosed herein, allowing the cosmetic composition to remain on the hair for a sufficient amount of time, and rinsing the cosmetic compositions from the hair. The cosmetic composition may be applied to the hair in a sequence with other compositions. For example, the cosmetic composition may be applied to the hair before shampooing the hair, after shampooing the hair, before conditioning the hair, and/or after conditioning the hair, etc. The compositions, however, are not required to be used in a sequence.

The methods may include applying an amount of the cosmetic composition onto the user's hair, for example, onto one or both hands, onto the hair, etc. The user's hair may already be wet or damp with extraneous water or extraneous water can be included after the cosmetic composition has already been applied to the hair. The extraneous water typically has a temperature of about 25° to 50° C. The cosmetic composition may be applied to the user's hand(s) or directly to the hair while the user is showering and/or bathing in water having a temperature of, e.g., 25° to 50° C. The cosmetic composition may optionally be rinsed from the user's hair.

Another unique aspect of the cosmetic compositions is that they may be used as a leave-on product. The cosmetic compositions can be applied to wet or damp hair and allowed to remain on the hair indefinitely, i.e., the cosmetic composition is not removed or rinsed from the hair prior to styling the hair.

In some cases, the cosmetic compositions are used in conjunction with additional hair-treatment compositions in a routine, for example, during an individual's normal showering/bathing routine. The cosmetic composition may be applied to the hair individually or may be combined with one or more additional compositions. Combining the compositions with one or more additional compositions (e.g., a shampoo, a conditioner, a rinse, etc.) can be useful for eliminating multiple steps from a routine. For instance, the cosmetic composition may be mixed with a shampoo (or conditioner) prior to application to the hair. In this case, the mixture of the shampoo (or conditioner) and the cosmetic composition are simultaneously applied to the hair during the cleansing or conditioning process and simultaneously rinsed from the hair. In at least one instance, however, the cosmetic composition is applied to hair after shampoo (or conditioner) has been rinsed from the hair. Alternatively, the cosmetic composition may be layered on top of (or lathered into) hair to which a shampoo (or conditioner) has already been applied or vice versa. For example, the composition may be applied to the hair and without rinsing it from the hair, a shampoo (or conditioner) is then subsequently applied to the hair. Alternatively, the shampoo (or conditioner) may be first applied to the hair and without rinsing the shampoo (or conditioner) from the hair, the cosmetic composition is also applied to the hair.

When used in conjunction with a shampoo and/or a conditioner, the cosmetic composition may be mixed or used with the shampoo and/or conditioner in a ratio of about 1:10 to about 10:1, about 1:5 to about 5:1, about 1:3 to about 3:1, about 1:2 to about 2:1, about 1:1 to about 4:1, about 1:1 to about 3:1, or about 1:1 to about 2:1 (cosmetic composition of the instant disclosure: shampoo/conditioner, etc.).

The cosmetic compositions of the instant disclosure may be allowed to remain on the hair for a minimum amount of time before being rinsed from the hair, but it is not necessary to allow the cosmetic composition to remain on the hair for an extended period of time. Conveniently, the cosmetic compositions can be applied and allowed to remain on the hair for a period of time that is typical for regular shampooing and/or conditioning. For example, the cosmetic composition (whether combined with another hair-treatment composition such as a shampoo or conditioner) may be applied to the hair and allowed to remain on the hair for a few seconds (1, 2, 3, or 5 seconds) up to about 1, about 2, about 5, about 10, about 15, about 20, about 25, or about 30 minutes.

When the cosmetic composition is not being mixed with another composition prior to application to the hair, the cosmetic composition may be applied to the hair immediately after or before the hair is treated with another composition (e.g., a shampoo and/or a conditioner). For example, the hair-treatment compositions may be applied to the hair within about a few seconds or 1, 2, 5, 10, or 20 minutes before or after a shampoo and/or a conditioner is applied to the hair.

### EMBODIMENTS

In certain embodiments, the cosmetic compositions of the instant disclosure have a lamellar network and include:
about 20 to about 84.5 wt. %, preferably about 30 to about 80 wt. %, more preferably about 60 to about 80 wt. %, of one or more glycols chosen from ethylene glycol, propylene glycol, butylene glycol, hexylene glycol, pentylene glycol, diethylene glycol, dipropylene glycol, 1,3 propanediol, polyethylene glycols, and a mixture thereof;
about 15 wt. % to about 50 wt. %, preferably about 18 wt. % to about 50 wt. %, more preferably about 18 wt. % to about 40 wt. %, of one or more trihydric alcohols having from 3 to 5 carbon atoms, such as glycerin, wherein the total amount of the one or more glycols of (a) is greater than the total amount of the one or more trihydric alcohols of (b);
about 0.1 to about 5 wt. %, preferably about 0.5 to about 4 wt. %, more preferably about 1 to about 3 wt. %, of one or more cationic surfactants, such as those selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof;
about 0.1 to about 5 wt. %, preferably about 0.1 to about 4 wt. %, preferably about 0.1 to about 3 wt. %, of one or more alkoxylated fatty alcohols, such as those alkoxylated with about 1 to about 50 moles of an alkylene oxide per mole of alkoxylated fatty alcohol; and
about 0.1 to about 5 wt. %, about 0.5 to about 4 wt. %, about 1 to about 3 wt. %, of one or more fatty alcohols, such as those having a carbon chain of 12 to 22 carbon atoms, wherein all percentages by weight are based on the total weight of the cosmetic composition.

In additional embodiments, the cosmetic compositions of the instant disclosure have a lamellar network and include:
about 20 to about 84.5 wt. %, preferably about 40 to about 80 wt. %, more preferably about 70 to about 80 wt. % of propylene glycol;
about 15 wt. % to about 50 wt. %, preferably about 18 wt. % to about 50 wt. %, more preferably about 18 wt. % to about 40 wt. %, of glycerin, wherein the total amount of the propylene glycol of (a) is greater than the total amount of the glycerin of (b);
about 0.1 to about 5 wt. %, preferably about 0.5 to about 4 wt. %, more preferably about 1 to about 3 wt. %, of one or more cationic surfactants, such as those selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof, the cationic surfactants preferably being behentrimonium chloride;
about 0.1 to about 5 wt. %, preferably about 0.1 to about 4 wt. %, preferably about 0.1 to about 3 wt. %, of alkoxylated fatty alcohols, wherein the alkoxylated fatty alcohols are chosen from ceteth compounds, ceteareth compounds, steareth compounds, laureth compounds, or a mixture thereof, the alkoxylated fatty alcohols preferably being alkoxylated stearyl alcohol
about 0.1 to about 5 wt. %, about 0.5 to about 4 wt. %, about 1 to about 3 wt. %, of one or more fatty alcohols, such as those having a carbon chain of 12 to 22 carbon atoms, wherein all percentages by weight are based on the total weight of the cosmetic composition
wherein the weight ratio of the combined weight of the one or more cationic surfactants of (c) and the one or more alkoxylated fatty alcohols of (d) relative to the combined weight of the one or more cationic surfactants of (c), the one or more alkoxylated fatty alcohols of (d), and the one or more of fatty alcohols of (e) is from about 0.2:1 to about 0.5:1, and wherein the cosmetic composition has a viscosity of about 10 to about 500 Pa·s at 24° C. and transitions to having a viscosity of less than 100 Pa·s at a temperature of 40° C., as measured with LV 04 Spindle on a Brookfield DV2T viscometer at a range of 1 to 10 rpm after 90 seconds, wherein all percentages by weight are based on the total weight of the cosmetic composition.

In further embodiments, a method for treating hair includes:
applying the cosmetic composition to hair and, optionally, rinsing the cosmetic composition from the hair;
optionally, mixing the cosmetic composition with shampoo prior to application to hair;
optionally, layering the cosmetic composition onto hair with a shampoo;
optionally, applying the cosmetic composition to hair after a shampoo has been rinsed from the hair;
optionally, layering the cosmetic composition onto hair with a conditioner;
optionally, mixing the cosmetic composition with a conditioner prior to application to hair; and/or
optionally, applying the cosmetic composition to hair after a conditioner has been rinsed from the hair.

### EXAMPLES

The following non-limiting examples are provided primarily for the purposes of elucidating the benefits and properties achieved by aspects of the invention.

### Example 1

Three non-limiting examples of cosmetic compositions (Exemplary Compositions A-C) were prepared in accordance with the disclosure described herein. Comparative Composition D was prepared in accordance with the method steps for producing Exemplary Compositions A-C. The formulations for Exemplary Compositions A-C and Comparative Composition D are provided below in Table 1.

TABLE 1

| | | | | | | D |
|---|---|---|---|---|---|---|
| | | | | | | (compar- |
| | | INCI US | A | B | C | ative) |
| (a) | Glycol | PROPYLENE | 76 | 76 | 75 | 76 |
| | | GLYCOL | | | | |
| (b) | Trihydric | GLYCERIN | 20 | 20 | 20 | 20 |
| | Alcohol | | | | | |
| (c) | Cationic | BEHEN- | 1.1 | 1.1 | 1.1 | 1.1 |
| | Surfactant | TRIMONIUM | | | | |
| | | CHLORIDE | | | | |
| | | AND/OR | | | | |
| | | CETRIMOUNIUM | | | | |
| | | CHLORIDE | | | | |
| (d) | Alkoxylated | STEARETH-20 | 0.2 | 0.2 | 0.2 | |
| | Fatty Alcohol | | | | | |
| (e) | Fatty Alcohol | MYRISTYL | | | | |
| | | ALCOHOL | | | | |
| | | CETEARYL | 1.9 | 1.9 | 1.9 | 1.9 |
| | | ALCOHOL | | | | |
| Ratio ((c) + (d))/((c) + (d) + (e)) | | | 0.41 | 0.41 | 0.41 | 0.37 |
| (f) | Cationic | POLY- | | 0.1 | 0.2 | |
| | polymer | QUATERNIUM-67 | | | | |
| | Monoalcohol | Ethanol | | | | |
| | Miscellaneous | FRAGRANCE | 1 | 1 | 1 | 1 |
| | | DICAPRYLYL | | | | |
| | | CARBONATE | | | | |
| | | ISOPROPYL | 0.2 | 0.2 | 0.2 | 0.2 |
| | | ALCOHOL | | | | |
| | | WATER | 0.04 | 0.04 | 0.04 | 0.04 |

| | | | | |
|---|---|---|---|---|
| Gel | Yes | Yes | Yes | Yes |
| Lamellar Structure | Yes | Yes | Yes | Yes |
| Temperature Activated | Yes | Yes | Yes | Yes |

### Example 2

The rheological properties of Exemplary Compositions A-C were assessed over a range of temperatures. For comparative purposes, the rheological properties of a traditional conditioner were also assessed under the same conditions as Exemplary Compositions A-C.

An AR G2 Rheometer was used with a Smart Swap™ Peltier plate temperature system to assess the rheological properties of Exemplary Compositions A-C and the traditional conditioner under stress of 0.1 to 0.5 Pa and frequency of 1 to 3 Hz as the temperature of the cosmetic compositions was increased from 20° C. to 50° C. Specifically, the AR G2 Rheometer and Smart Swap™ Peltier plate temperature system was used to measure the storage modulus (G), which describes the level of solid-state behavior exhibited by the composition, and to measure the loss modulus (G′), which describes the level of liquid-state behavior exhibited by the composition.

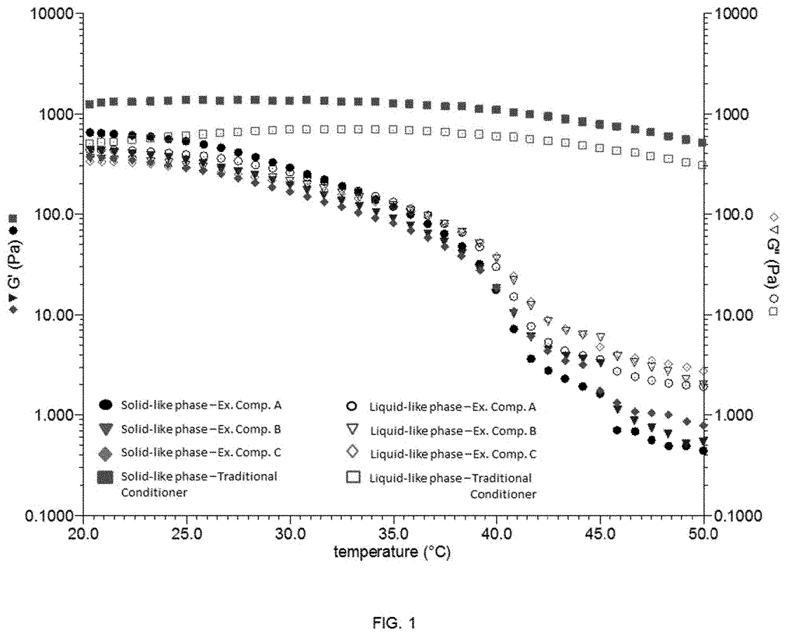

FIG. 1 is a graph showing the results of an oscillatory temperature sweep experiment measuring storage modulus and loss modulus of Exemplary Compositions A-C and the traditional conditioner, with a stress of 0.5 Pa and frequency of 3 Hz as the temperature of the cosmetic compositions was increased from 20° C. to 50° C. The solid plot points in FIG. 1 represent the level storage modulus at a specific temperature. The outlined plot points in FIG. 1 represent the level of loss modulus at a specific temperature.

As is shown in FIG. 1, Exemplary Composition A exhibited a greater level of storage modulus than loss modulus from 20° C. to about 32° C. indicating dominance of solid-like behavior. From about 34° C. to about 50° C., exemplary Composition A exhibited a greater level of loss modulus than storage modulus indicating dominance of liquid-like behavior. Exemplary Composition A exhibited a rheological phase transition between 32° C. and 34° C. as the Exemplary Composition transitioned from exhibiting greater solid-like behavior to exhibiting greater liquid-like behavior.

Exemplary Compositions B and C have a rheological phase transition over a temperature range that is lower than that of Exemplary Composition A. Without being limited to any one theory, the inventors believe that the addition of a cationic polymer, such as POLYQUATERNIUM-67, can lower the temperature range of the rheological phase transition (e.g. from a viscoelastic solid to a viscoelastic liquid).

The traditional conditioner had a greater level of storage modulus than loss modulus over the whole temperature range of 20° C. to 50C°, indicating that the traditional conditioner did not exhibit a rheological phase transition.

### Example 3

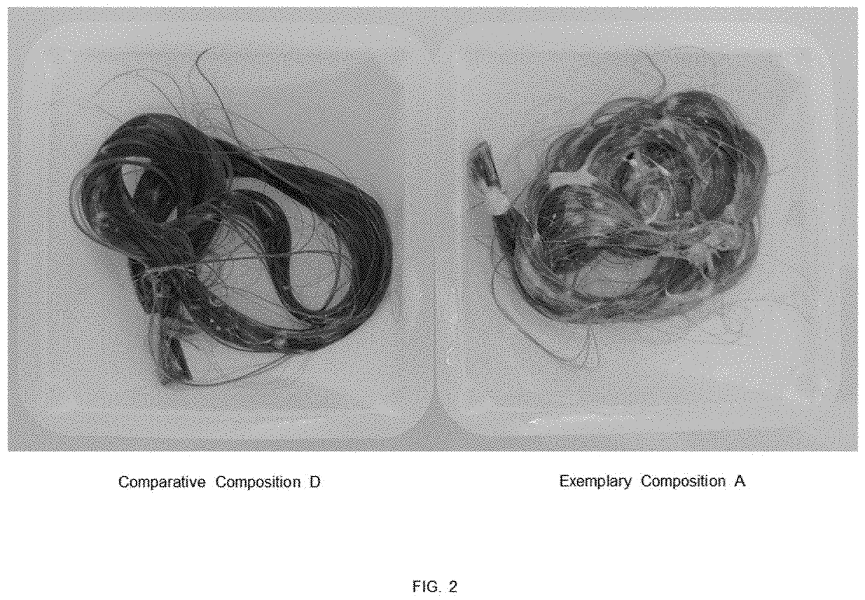

Exemplary Composition A and Comparative Composition D were applied to medium bleached hair swatches and assessed for foamability. Prior to the application of Exemplary Composition A and Comparative Composition D, the hair swatches were each washed with 1 gram of shampoo. While the hair swatches were damp, 1 gram of Exemplary Composition A and 1 gram of Comparative Composition D were applied to separate hair swatches. Each of the hair swatches were swirled in a plastic weigh boat 20 times. As shown in FIG. 2, significantly more foam was produced by Exemplary Composition A as compared to Comparative Composition D.

The term “INCI” is an abbreviation of International Nomenclature of Cosmetic Ingredients, which is a system of names provided by the International Nomenclature Committee of the Personal Care Products Council to describe personal care ingredients.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

All components and elements positively set forth in this disclosure can be negatively excluded from the claims. In other words, the cosmetic compositions of the instant disclosure can be free or essentially free of all components and elements positively recited throughout the instant disclosure.

In some instances, the cosmetic compositions of the present disclosure may be substantially free of non-incidental amounts of the ingredient(s) or compound(s) described herein. A non-incidental amount of an ingredient or compound is the amount of that ingredient or compound that is added into the cosmetic composition by itself. For example, a cosmetic composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the composition includes both components (or the composition includes more than two components that overlap), an overlapping compound does not represent more than one component. For example, steareth-20 may be characterized as both an alkoxylated fatty alcohols and a nonionic surfactant. If a particular composition includes both an alkoxylated fatty alcohols and a nonionic surfactant, steareth-20 will serve only as the alkoxylated fatty alcohols or only as the alkoxylated fatty alcohols (steareth-20 does not serve as both the alkoxylated fatty alcohols and nonionic surfactant).

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

The expression “one or more” means “at least one” and thus includes individual components as well as mixtures/combinations.

Other than in the operating examples, or where otherwise indicated, all numbers expressing quantities of ingredients and/or reaction conditions may be modified in all instances by the term “about,” meaning within +/−5% of the indicated number.

The term “treat” (and its grammatical variations) as used herein refers to the application of the compositions of the present disclosure onto the surface of keratinous substrates such as hair on a user's head and/or body.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. All of the components set forth herein may be optionally included or excluded from the compositions/method/kits. When excluded, the compositions/methods/kits may be free or essentially free of the component. For example, a particular composition may be free or essentially free of silicones.

What is claimed is:

1. A substantially anhydrous cosmetic composition having a lamellar network comprising:
(a) about 20 to about 84.5 wt. % of one or more glycols chosen from ethylene glycol, propylene glycol, butylene glycol, hexylene glycol, pentylene glycol, diethylene glycol, dipropylene glycol, 1,3 propanediol, polyethylene glycols, and a mixture thereof;
(b) about 15 wt. % to about 50 wt. % of one or more trihydric alcohols having from 3 to 5 carbon atoms;
wherein the total amount of the one or more glycols of (a) is greater than the total amount of the one or more trihydric alcohols of (b);
(c) about 0.1 to about 5 wt. % of one or more cationic surfactants;
(d) about 0.1 to about 5 wt. % one or more alkoxylated fatty alcohols; and
(e) about 0.1 to about 10 wt. % of one or more fatty alcohols,
wherein all percentages by weight are based on the total weight of the cosmetic composition.

2. The cosmetic composition of claim 1 wherein the weight ratio of the combined weight of the one or more cationic surfactants of (c) and the one or more alkoxylated fatty alcohols of (d) relative to the combined weight of the one or more cationic surfactants of (c), the one or more alkoxylated fatty alcohols of (d), and the one or more of fatty alcohols of (e) is from about 0.2:1 to about 0.5:1.

3. The cosmetic composition of claim 1, wherein the composition has a viscosity of about 10 to about 500 Pa·s at 24° C. and a viscosity of less than 100 Pa·s at a temperature of 40° C., as measured with LV 04 Spindle on a Brookfield DV2T viscometer at a range of 1 to 10 rpm after 90 seconds.

4. The cosmetic composition of claim 1, wherein the one or more glycols comprises propylene glycol.

5. The cosmetic composition of claim 1, wherein the one or more tryihdyric alcohols are chosen from glycerol, a butanetriol, a pentanetriol, and a mixture thereof.

6. The cosmetic composition of claim 5, wherein the one or more tryihdyric alcohols are chosen from glycerol, 1,2,3-butanetriol, 1,2,4-butanetriol, 1,2,5-pentanetriol, and a mixture thereof.

7. The cosmetic composition of claim 1, wherein the one or more trihydric alcohols comprises glycerin.

8. The cosmetic composition of claim 1, wherein the one or more cationic surfactants are chosen from quaternary ammonium compounds, amidoamines, or a mixture thereof.

9. The cosmetic composition of claim 1, wherein the one or more cationic surfactants are chosen from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

10. The cosmetic composition of claim 1, further comprises:
(f) about 0.01 to about 6 wt. % of one or more cationic polymers.

11. The composition of claim 1, wherein the fatty alcohol group of the one or more alkoxylated fatty alcohols has a carbon chain of greater than 8 carbon atoms.

12. The composition of claim 11, wherein the one or more alkoxylated fatty alcohol are alkoxylated with about 1 to about 100 moles of an alkylene oxide per mole of alkoxylated fatty alcohol.

13. The composition of claim 1, wherein the one or more fatty alcohols has a carbon chain of 12 to 22 carbon atoms.

14. The cosmetic composition of claim 1, wherein the one or more fatty alcohols are chosen from decyl alcohol, undecyl alcohol, dodecyl alcohol, myristyl alcohol, cetearyl alcohol, cetyl alcohol, stearyl alcohol, isostearyl alcohol, isocetyl alcohol, behenyl alcohol, linalool, oleyl alcohol, cis-4-t-butylcyclohexanol, isotridecyl alcohol, myricyl alcohol, and a mixture thereof.

15. The cosmetic composition of claim 1 being substantially free of monoalcohols having a carbon chain of 1 to 8 carbons.

16. The cosmetic composition of claim 1 being substantially free of water.

17. The cosmetic composition of claim 1 being substantially free of silicone.

18. A substantially anhydrous cosmetic composition having a lamellar network comprising:
(a) about 20 to about 84.5 wt. % of propylene glycol;
(b) about 15 wt. % to about 50 wt. % of glycerin;
wherein the total amount of the propylene glycol of (a) is greater than the total amount of the glycerin of (b);
(c) about 0.1 to about 5 wt. % of one or more cationic surfactants;
(d) about 0.1 to about 5 wt. % alkoxylated fatty alcohols, wherein the alkoxylated fatty alcohols are chosen from ceteth compounds, ceteareth compounds, steareth compounds, laureth compounds, or a mixture thereof;
(e) about 0.1 to about 10 wt. % of one or more fatty alcohols,
wherein the weight ratio of the combined weight of the one or more cationic surfactants of (c) and the one or more alkoxylated fatty alcohols of (d) relative to the combined weight of the one or more cationic surfactants of (c), the one or more alkoxylated fatty alcohols of (d), and the one or more of fatty alcohols of (e) is from about 0.2:1 to about 0.5:1,
the composition has a viscosity of about 10 to about 500 Pa·s at 24° C. and a viscosity of less than 100 Pa·s at a temperature of 40° C., as measured with LV 04 Spindle on a Brookfield DV2T viscometer at a range of 1 to 10 rpm after 90 seconds, and
all percentages by weight are based on the total weight of the cosmetic composition.

19. A method for treating hair comprising applying the cosmetic composition of claim 1 to hair and, optionally, rinsing the cosmetic composition from the hair.

20. The method of claim 19 further comprising:
mixing the cosmetic composition of claim 1 with shampoo prior to application to hair;
layering the cosmetic composition of claim 1 onto hair with a shampoo;
applying the cosmetic composition of claim 1 to hair after a shampoo has been rinsed from the hair;
layering the cosmetic composition of claim 1 onto hair with a conditioner;
mixing the cosmetic composition of claim 1 with a conditioner prior to application to hair; and/or
applying the cosmetic composition of claim 1 to hair after a conditioner has been rinsed from the hair.
